(19) United States
(12) Patent Application Publication
(10) Pub. No.: US 2026/0306940 A1
(43) Pub. Date: Oct. 8, 2026

(54) SYSTEMS AND METHODS FOR PROVIDING HAPTIC FEEDBACK TO DHH PERSONS

(71) Applicant: Morat Gaming LLC, Sherborn, MA (US)

(72) Inventors: Christopher I. Lehrich, Sherborn, MA (US); Kevin J. Nolan, Sherborn, MA (US)

(73) Assignee: Morat Gaming LLC, Sherborn, MA (US)

(21) Appl. No.: 19/481,588

(22) PCT Filed: May 23, 2024
(86) PCT No.: PCT/US2024/030738

§ 371 Date: Nov. 5, 2025

Related U.S. Application Data
(60) Provisional application No. 63/504,579, filed on May 26, 2023.

(51) Int. Cl. A63F 13/285; A63F 13/79; G06F 3/01; G09B 21/00

(52) U.S. Cl. CPC A63F 13/285; A63F 13/79; G06F 3/016; G09B 21/009

(57) ABSTRACT

A system and method of providing haptic feedback in a virtual environment generates a virtual instance such as a game level and an interaction within the virtual instance. The interaction includes a first interaction parameter and may be an event such as an explosion within the virtual instance. The system determines a spatial relation between a player avatar and the interaction to identify an environment between the player avatar and the interaction. A total situational state including the virtual instance and the interaction is generated. At least one actuator instruction is generated based at least in part on the total situational state and sent to a wearable haptic rig. The at least one actuator instruction is applied to at least one haptic motor of the wearable haptic rig to actuate the at least one haptic motor and provide haptic feedback to the player.

### CROSS-REFERENCE TO RELATED APPLICATIONS DATA

This application claims the benefit of U.S. Provisional Application No. 63/504,579 entitled “SYSTEMS AND METHODS FOR PROVIDING HAPTIC FEEDBACK TO DHH PERSONS” and filed May 26, 2023, which is hereby incorporated by reference.

### FIELD OF TECHNOLOGY

The present disclosure relates generally to haptic feedback devices for DHH persons.

### BACKGROUND

Current interactive technologies such as videogames, movies, and TV shows do not create an immersive experience for Deaf and hard-of-hearing (DHH) persons. The use of subtitles and vibration in controllers has previously been used to try and bridge the gap in experience. However, these substitutes fail to replace sound in recreating the immersive experience. Instead, they use a second medium such as touch to enhance a virtual soundscape.

In prior art systems, an audio card would locate and play a sound file relevant to an interaction. In the example of an explosion, a pre-recorded sound file of an explosion would be played. To improve immersion, prior art systems calculated how the sound would play upon reaching the player character based on environmental factors. For example, if a player character was standing in a stone hallway, the explosive sound could be echoed to provide a 3D sound experience. In addition, some prior art systems would vibrate a game controller in addition to the echoed sound to provide a sense of realism.

Traditional methods relied on a hearing individual's situational awareness constructed through continuous audio feedback to create the immersive experience. These methods, such as head-related transfer functions (“HRTF”), recreate an instance of a hearing individual's auditory system for receiving sound. HRTF uses the frequency and phase response of the average hearing individual (e.g., the distance between the ears, detectable audio range, etc.) for calculating the situational awareness. However, HRTF relies on a hearing individual continuously constructing their situational awareness in large part by combining and interpreting analog aural signals arriving distinctly at two ears. Thus, previous attempts at constructing virtual situational awareness presumed that any additional feedback, such as vibration of a game controller, would be added to the situational awareness “picture” already created by audio signals. However, these methods fail to inform the individual of changes to their surroundings when audio signals are not creating the “picture”, as with a DHH person.

The use of 3D surround-sound to improve the immersive experience has only widened the gap in experiences for DHH persons. For example, current technologies may use sound to provide a player the sensation of hearing an enemy approaching from the high-rear-left, causing the player to turn sharply to face the sound and be ready to battle the enemy at once. These experiences are currently inaccessible to DHH persons. Thus, a need remains for offering an immersive experience to DHH persons on par with the audio experience.

### SUMMARY

The present disclosure provides a haptic feedback system for providing haptic feedback to DHH persons and creating an immersive media experience. A media player such as a video game console may create a virtual instance such as a game level. An interaction may be generated such as an explosion within the virtual instance. The haptic feedback system may calculate the location of a player avatar compared to the interaction. The haptic feedback system may modulate haptic feedback provided to the player using a wearable haptic rig based at least in part on determined parameters defined by the virtual instance's environment. As a result, the haptic feedback system may provide a DHH player haptic feedback such as a tactile sensation indicating a location and intensity of the interaction. The tactile sensation may be modulated using the determined parameters to provide an immersive experience in place of prior art sound systems.

The media player may generate an environmental parameter such as an environmental effect based on the determined parameters defined by the virtual instance's environment. For example, the environmental parameter may be a distortion effect, an obstruction effect, or an occlusion effect applied by the environment of the virtual instance. A first interaction parameter generated by the interaction may be modified by the environmental parameter to generate a second interaction parameter.

The haptic feedback system may include a signals acquisition unit for intercepting the first interaction parameter. A data interceptor may receive the first interaction parameter and store it in a memory. The first interaction parameter and the second interaction parameter may be sent to a data comparator for comparing the first interaction parameter from the data interceptor and the second interaction parameter. The data comparator matches an identifier in the first interaction parameter and the second interaction parameter to compare and isolate the changes made by the environmental parameter. The data comparator uses the changes to create a total situational state. The total situational state creates a “picture” of how each signal produced by the interaction at a specified location and time is affected by the environmental parameter before reaching the player avatar.

A microprocessor receives the total situational state. The microprocessor may classify and prioritize at least one parameter of the total situation state from the signals acquisition unit. The classification may determine the importance of an interaction based on its location relative to the player avatar, intensity, and “threat” within the level or virtual instance. The classified and prioritized parameters are filtered by a user situational filter to determine which interactions should be provided to the player. The user situational filter may be created using calibration settings specific to the player. For example, a virtual instance may include multiple interactions such as explosions occurring at the same time throughout a level. Providing haptic feedback from all the interactions at the same time could overwhelm or disorient a player.

The user situational filter may be applied to remove excessive “noise” from the total situational state before the microprocessor compiles and outputs actuator instructions to a wearable haptic rig for providing haptic feedback to the player.

A wireless or wired receiver may receive and transmit the actuator instructions to a haptic rig processor. At least one haptic motor may be actuated to provide haptic feedback to the player to create the immersive experience.

### BRIEF DESCRIPTION OF THE DRAWINGS

Embodiments of devices, systems, and methods are illustrated in the figures of the accompanying drawings which are meant to be exemplary and non-limiting, in which like references are intended to refer to like or corresponding parts, and in which:

FIG. 1 illustrates an embodiment of a haptic feedback rig according to the disclosure;

FIG. 2 illustrates a block diagram of an embodiment of a haptic feedback system according to the disclosure;

FIG. 3 illustrates a flow chart of an embodiment of the haptic feedback system generating a total situational state according to the disclosure;

FIG. 4A is an illustrative embodiment of the media player processor of the haptic feedback system generating a first interaction parameter according to the disclosure;

FIG. 4B is an illustrative embodiment of the interaction processor of the haptic feedback system generating an environmentally affected interaction information based on the first interaction parameter of FIG. 4A according to the disclosure;

FIG. 4C is an illustrative embodiment of the data interceptor of the haptic feedback system generating an original interaction information based on the first interaction parameter of FIG. 4A according to the disclosure;

FIG. 4D is an illustrative embodiment of the data comparator of the haptic feedback system generating a total situational state based on the environmentally affected interaction information of FIG. 4B and the original interaction information of FIG. 4C according to the disclosure; and

FIG. 5 is a block diagram illustrating a method of providing a haptic based immersive experience according to the disclosure.

### DETAILED DESCRIPTION

The detailed description of aspects of the present disclosure set forth herein makes reference to the accompanying drawings, which show various embodiments by way of illustration. While these various embodiments are described in sufficient detail to enable those skilled in the art to practice the disclosure, it should be understood that other embodiments may be realized and that logical and mechanical changes may be made without departing from the spirit and scope of the disclosure. Thus, the detailed description herein is presented for purposes of illustration only and not of limitation. For example, the steps recited in any of the method or process descriptions may be executed in any order and are not limited to the order presented. Moreover, references to a singular embodiment may include plural embodiments, and references to more than one component may include a singular embodiment.

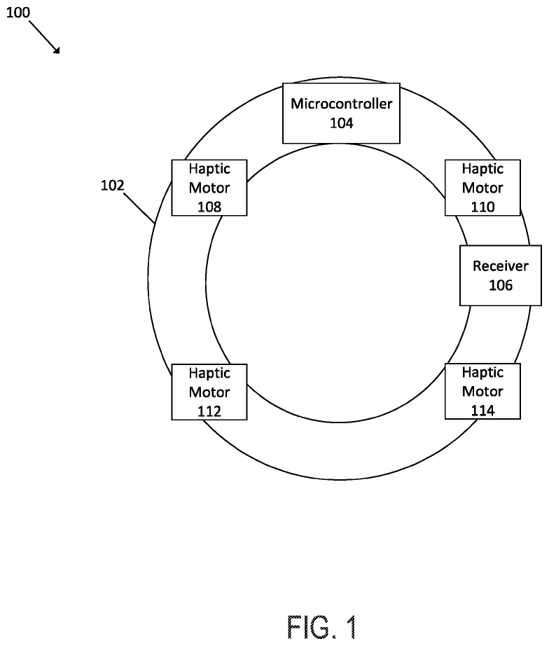

FIG. 1 illustrates an embodiment of a haptic feedback rig 100 according to the disclosure. A collar 102 includes a microcontroller 104 for receiving actuator instructions from a receiver 106. The microcontroller 104 reads the actuator instructions and actuates the haptic motors 108, 110, 112, 114 to provide a haptic feedback to a player.

The haptic feedback rig 100 may communicate with a haptic feedback system as disclosed herein including a media player such as a game console. The haptic feedback system generates and transmits to the haptic feedback rig 100 actuator instructions for actuating the haptic motors 108, 110, 112, 114. The media player may generate a virtual instance such as a videogame level or a movie. An interaction or event may occur as part of the virtual instance. The haptic feedback system processes the interaction to generate the actuator instructions and provide haptic feedback to the player. The haptic feedback provided by the haptic motors 108, 110, 112, 114 creates an immersive experience in place of a traditional soundscape provided using audio signals.

In an illustrative embodiment, the actuator instructions may cause a single haptic motor of haptic motors 108, 110, 112, 114 to actuate and provide an individual pulse of a determined intensity relating to an interaction. Depending on the type and duration of the interaction, the actuator instructions may actuate any combination of haptic motors 108, 110, 112, 114 to provide cascades or chain pulses of determined intensities. By way of example, a first pulse may be provided to the player by actuating haptic motor 108. Following the first pulse, or overlapping with the first pulse, a second pulse may be provided by actuating haptic motor 110. The second pulse may follow the first pulse within microseconds. A third pulse may follow the second pulse by actuating haptic motor 112 and a fourth pulse may be provided by actuating haptic motor 114. The actuator instructions thus simulate a cascading effect or other immersive experience to provide the player haptic feedback relating to the interaction. The actuator instructions may cause each pulse to be of the same intensity, a weakening intensity, or a strengthening intensity. For example, the first pulse may be stronger than the second pulse.

As a result, if the player is a DHH person or playing without sound, the player may experience immersion into and awareness of their surroundings in the virtual instance.

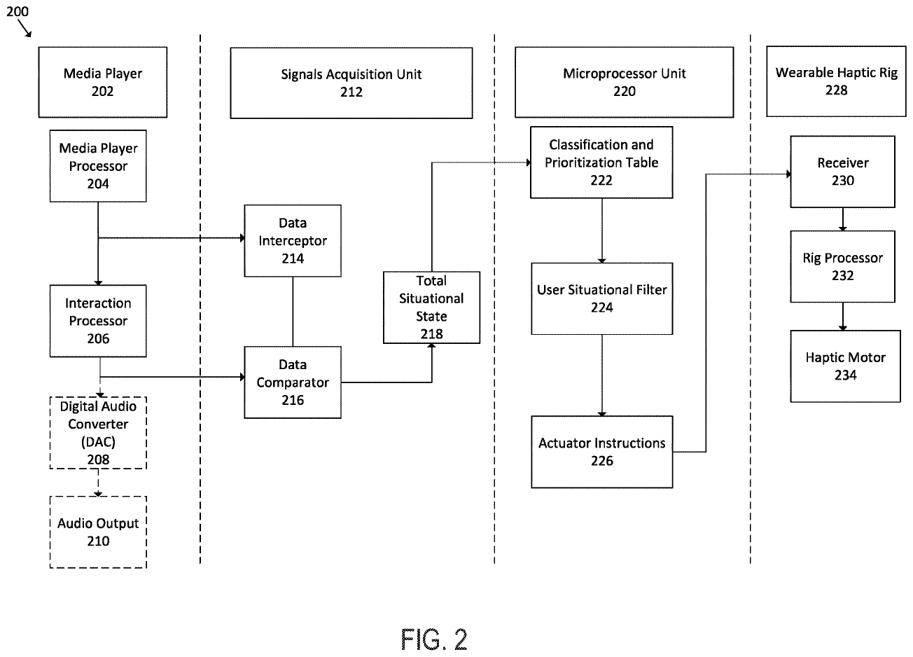

FIG. 2 illustrates a block diagram of an embodiment of a haptic feedback system 200 according to the disclosure. A media player 202 such as a game console may include a media player processor 204 for generating a virtual instance such as a video game level. As part of the virtual instance, the media player 202 may generate an interaction within the virtual instance. These interactions may range from conversing with Non-Playable Characters (“NPC”) within the virtual instance to an event such as an explosion or a fight. Each of these interactions may affect a player. The player thus requires awareness of the interaction, provided by the haptic feedback system 200 as haptic feedback.

The media player processor 204 may generate a first interaction parameter as a data packet for transmitting to an interaction processor 206. The first interaction parameter may include information detailing the generated interaction such as the type of interaction and the location of the interaction. The interaction processor 206 may, in an embodiment, include an audio card located on or connected to the media player processor 204 or connected to a motherboard of the media player 202. The interaction processor 206 is responsible for applying any environmental effects to the first interaction parameter based on the environment of the virtual instance. The environmental effect may be output as an environmental parameter for modifying or enhancing the first interaction parameter to generate a second interaction parameter. For example, the environmental parameter may be a distortion effect, an obstruction effect, or an occlusion effect applied by the environment of the virtual instance.

In an embodiment, vibration from an explosion may be defined with the first interaction parameter. As a person of ordinary skill in the art understands, sound is generated by vibrations traveling through a fluid such as air. The vibration caused by the explosion may be interpreted as an impact/touch against the skin by a DHH person. A hearing individual may also interpret the vibrations as a sound generated by the explosion. The vibration may be modified by the environmental parameter by applying a muffling effect to the first interaction parameter to generate the second interaction parameter including a muffled vibration. The muffled vibration could be generated to replicate the real-world physics of the explosion occurring on the other side of a door from the player avatar.

In order to generate and define the environmental parameters, the media player processor 204 and the interaction processor 206 generate a trace (such as by “wave tracing” or “raytracing”) of the interaction as it approaches the player avatar. The trace may include any reflection, obstruction or occlusion caused by the virtual environment to the interaction as it travels from its origin point in the virtual instance towards the player avatar. These environmental parameters may create distortions or echoes in how the player experiences the interaction when it reaches the player avatar. As a result, an interaction with a clear line-of-sight to the player avatar may reach the player avatar more quickly and with fewer environmental effects applied to it than an interaction in another room or located behind an object in the environment. The trace generates the environmental parameter as at least one environmental effect to be applied to the interaction.

An example of a scenario requiring a distortion effect may include an explosion which occurs down a long stone hallway. As the vibrations from the explosion travel down the stone hallway, the vibrations may bounce off the stone walls causing an echo effect to be applied. The echo may distort the original vibration such that it reaches the player avatar differently than would an explosion occurring in an empty field. The path of the vibrations may be traced to generate the environmental parameter, including the echo as the environmental effect. When the vibrations reach the player avatar, the player must be provided feedback simulating the echo to create a feeling of immersion.

A signals acquisition unit 212 may include a data interceptor 214 for receiving the (pre-trace) first interaction parameter from the media player processor 204 and a data comparator 216 for receiving the second interaction parameter and the environmental parameter from the interaction processor 206. The data interceptor 214 intercepts and stores the first interaction parameter including, but not limited to, the nature of the interaction, such as if it is a vehicle, an explosion, a weather effect, or a notification; the threat, i.e., within the virtual instance how “dangerous” is it to the player avatar, intensity, such as strength of the interaction; location, i.e., where the interaction occurred relative to the player avatar; velocity, i.e., the speed at which the interaction is approaching the player avatar; and acceleration, i.e., any changes in the velocity as the interaction approaches the player avatar. The data interceptor may output the first interaction parameter or an original interaction parameter for comparing to the second interaction parameter by the data comparator 216.

Interaction processors, such as audio cards, produce an environmentally affected parameter but may not store the changes made to produce the environmental effect. Traditional DAC methods focus on the sound produced and so do not necessarily save the trace and environmental parameter for indicating what the changes to the first interaction parameter were. Saving the trace may require additional processing power or storage which, when the only concern is outputting a modified sound, are unnecessary. Thus, to identify these changes, the data comparator 216 receives the first interaction parameter of the original interaction parameter from the data interceptor 214 and the second interaction parameter from the interaction processor 206. As a person of ordinary skill in the art would understand, the signals acquisition unit 212 may be part of the interaction processor 206 without departing from the disclosure. For example, the interaction processor 206 may be modified to include storage for saving the first interaction parameter as a data interceptor 214 without departing from the disclosure. Similarly, the interaction processor may be modified to store the changes made to generate the second interaction parameter as a data comparator 216 without departing from the disclosure.

The data comparator 216 outputs a total situational state 218 based on the comparison. The total situational state 218 may include the first interaction parameter, the environmental parameter as identified by the data comparator 216, and the second interaction parameter. The total situational state 218 may include the virtual instance and construct a 3D instance of how the interaction would be received by the player avatar at a defined location. The signals acquisition unit 212 may generate the total situational state 218 without the use of an audio signal such as used by digital-audio-converters (DAC) 208. DAC 208 requires a large amount of processing power and can slow down processing of an interaction. Additionally, where the player is a DHH person or has the sound turned off, the processing power consumed to generate the audio signals for the DAC 208 is wasted. The DAC 208 typically only receives signals for producing an environmentally affected sound generated by the interaction or for translating the sound back into a digital signal. The signals acquisition unit 212 improves the speed of constructing the total situational state 218 by obviating the need for the DAC 208 to create a soundscape using an audio output 210 in the process.

An embodiment of the total situational state 218, continuing the example of an explosion, may be created by compiling similar explosion signals generated by the media player processor 204. Each signal may be given an identifier (such as “A-” to indicate the signal is part of interaction A) and a series of tags. The tag or tags, depending on the interaction, are output as the first interaction parameter in a data packet by the media player processor 204. A tag may include the signal format such as a .WAV file. Another tag may be an accessibility tag such as if the player has turned on subtitles. The accessibility tag may, in this example, add a subtitle file to the first interaction parameter. Other tags may include a location, the type of surroundings such as that the player avatar is in a stone hallway, the base (original) strength of the interaction, the velocity, etc.

Each tag for a single interaction may include the same identifier. A first interaction parameter may include one or more interactions; thus the identifier may be used to distinguish the tags from each other. If two interactions, such as an explosion and the sound of something breaking (for example, a barrel near the explosion) occur at the same time/location, they may be transmitted as part of the first interaction parameter. The explosion interaction signals may include an identifier of “explosion-” whereas the barrel interaction signals may include an identifier of “barrel-”. As a result, the total situational state 218 may group similar signals together using the identifiers included in the first interaction parameter.

An illustrative embodiment of a first interaction parameter may be:
[A-0001] electronic explosion [A-0002] elecexplo.wav [A-0003] “electronic explosion” [A-0004 ] 30 meters [A-0005] sphere [A-0006] static [A-0007] diminish 30% [A-0008] 17.15.−257

Identifier A may be used to group signals for an electronic explosion within the virtual instance. The identifier may be defined at [A-0001] as “electronic explosion.” A source file [A-0002] “elecexplo.wav” is tagged. If subtitles are turned on, [A-0003] “electronic explosion” may be displayed on a graphical user interface connected to the media player 202. The explosion may have [A-0004] flagging a “danger” radius of 30 meters in [A-0005] a sphere which [A-0006] doesn't move, i.e., is static once generated. The force of the explosion [A-0007] may diminish at a 30% rate over time. The location of the explosion [A-0008] may be at x-coordinate 17, y-coordinate 15, and z-coordinate −257 in the virtual instance.

The interaction processor 206, on receiving the first interaction parameter, completes the trace to calculate potential distortions, and applies the identified distortion(s), obstruction(s), and occlusion(s) to generate the second interaction parameter. The signal is “bounced” off every available surface, with all echoes, obstructions, occlusions, and partial absorptions by soft surfaces applied as the environmental effect(s). The second interaction parameter may include the same identifiers as the first interaction parameter with the tags modified by the environmental effect(s). Similar or matching identifiers are grouped for comparing the first interaction parameter to the second interaction parameter. The comparison may be used to identify any changes made by the virtual environment to the interaction.

As described hereinbefore, DAC systems for producing a soundscape do not require detailed information on the environmental effects, such as how much distortion, obstruction, or occlusion was applied. The DAC 208 and the audio output 210 are only concerned with the end result of the second interaction parameter which may already include the environmental effects. Generating the total situational state 218 requires the detailed information on the environmental effect. To capture this information, the data interceptor 214 stores the first interaction parameter and the data comparator 216 determines the environmental effects. The data interceptor 214 and the data comparator 216 may be implemented as elements of the signals acquisition unit 212 to retain the detailed information on the environmental effects and generate the total situational state 218. The signals acquisition unit 212 may be part of the media player 202 or a microprocessor unit 220 without departing from the disclosure.

The total situational state 218 may be received by the microprocessor unit 220. The microprocessor unit 220 may apply a classification and prioritization table 222 to evaluate the interaction(s) in the total situational state 218 based on their importance to the player. The classification and prioritization table 222 may utilize the tags included in the first interaction parameter and the second interaction parameter to rank the interaction signals. The classification and prioritization table 222 may determine whether a player can detect the interaction or if the interaction is dangerous enough to require providing the player notice. To avoid overwhelming a player, outputting haptic feedback for every interaction may not be desired.

Thus, if multiple interactions are occurring at once, for example on a battlefield, providing the player haptic feedback for every explosion, vehicle movement, gunfire, NPC, and weather effect may overwhelm and disorient the player instead of creating an immersive experience. The polyphony, the number of interactions a system may generate at a time, ranges widely across different media systems. A media player 202 may be able to output 50 interactions at once or 5,000. However, the player may not be able to digest and understand haptic feedback from all 50 interactions or all 5,000 interactions depending on the intensity of the haptic feedback. As an illustrative example, if 100 people are shouting in a crowded room, determining what an individual said may be impossible, whereas if only 20 people are shouting it may be possible. The interactions occurring in the virtual instance may be ranked and filtered by the classification and prioritization table 222 to prevent overwhelming the player. In the example of the crowded room, the classification and prioritization table 222 may identify the 80 or so voices which would be unintelligible or are unimportant and filter them out, leaving only 20 voices for the player to respond to.

The classified and prioritized parameters of the total situational state 218 are filtered using a user situational filter 224 tracking which interaction(s) the player is aware of. The user situational filter 224 may be based in part on a calibration profile specific to the player. Each individual has their own level of spatial awareness, reaction speed, etc. By applying the user situational filter 224 to the classified and prioritized total situational state 218, a player-specific cutoff may be applied. In this way, the player is not bombarded with numerous interactions outside their awareness. For example, if an interaction takes place behind a closed door and the player is determined to not be aware of the interaction, the classification and prioritization table 222 may rank the interaction below other interactions occurring at the same time. The user situational filter 224 may filter out the interaction based on the player's calibrated awareness levels until the player becomes aware of it.

A player with a high level of spatial awareness may notice the interaction behind the closed door despite their distance if no other interactions are occurring at the same time. The microprocessor unit 220 translates the classified and prioritized parameters of the total situational state 218 into actuator instructions 226 for sending to the wearable haptic rig 228. As a result, the player will not be overwhelmed by haptic feedback drowning out “important” interactions occurring in the virtual instance. To continue the example of the crowded room above, if a player's individual awareness level is such that they would only be able to digest 15 voices, the user situational filter 224 may filter out the 5 lowest ranked voices of the 20 remaining.

The user situational filter 224 may include a player profile based at least in part on a calibration profile specific to the player. DHH players may perceive and utilize situational-awareness information in distinctive, partly-unique ways. In addition, ongoing use of sensory-alteration devices (eyeglasses, hearing aids, etc.) trains a user's brain, such that over time the device may change its efficiency for that user (e.g., people with mild hearing loss who use hearing aids must periodically re-calibrate them as their ears and brains adapt to the altered soundscape). The user situational filter 224 may be updated using the calibration profile during calibration sessions or during a play session. The player may be monitored, for example using haptic feedback, for various parameters such as their reaction time during play to fine tune the calibration profile and update the user situational filter 224. The user situational filter 224 may account for a DHH player's individual situational awareness in translating the total situational state 218 into the actuator instructions 226.

The actuator instructions 226 are sent to the wearable haptic rig 228 for providing haptic feedback to the player. The wearable haptic rig 228 may include a receiver 230, such as a wireless or Bluetooth® receiver or a wired connection, for receiving the actuator instructions 226 from the microprocessor unit 220. A rig processor 232 applies the actuator instructions 226 to actuate at least one haptic motor 234 for providing haptic feedback to the player. The haptic feedback may capture any environmental effect applied by the virtual instance's environment to the interaction to provide the player an immersive experience. In an embodiment, the microprocessor unit may be mounted on the wearable haptic rig 228 and directly send the actuator instructions 226 to the at least one haptic motor 234. As a result, the player may experience an immersive interaction provided by the haptic feedback including the nature, intensity, and location, among other things, of the interaction in the virtual instance.

In an illustrative embodiment of the haptic feedback system 200 the media player 202 may generate a game level with the player avatar standing in a stone hallway. An explosion may take place at a location in the virtual instance such as 50 meters along the stone hallway directly in front of the player. The impact/force from the explosion is analyzed along with environmental factors such as any distortion or echo caused by the stone hallway on the explosion's shockwave. The signal acquisition unit 212 processes the stone hallway virtual instance, the explosion, and the location of the player to create the total situational state 218.

The microprocessor unit 220 receives the total situational state 218 and classifies and prioritizes the explosion and any sub-interactions, such as objects breaking at the explosion site or enemy avatar reactions, such that the interactions are prioritized and handled appropriately. For example, the explosion may be more important to the player than the objects breaking at the explosion site. The user situational filter 224 is applied based in part on the calibration profile to filter out the interactions the player isn't aware of or which would overwhelm the player. For example, if the player is determined to have a low situational awareness because another interaction has taken their attention or their reaction speed is determined to be below a predetermined threshold, the player may not be aware of or able to react to the enemy avatars. Therefore, the enemy avatar reactions may not be provided to the player as haptic feedback. The microprocessor unit 220 translates the filtered interaction(s) from the total situational state 218 into actuator instructions 226 and sends the actuator instructions 226 to the wearable haptic rig 228.

The wearable haptic rig 228 receives the actuator instructions 226 relating to the explosion using the receiver 230. The rig processor 232 processes the actuator instructions 226 and disseminates the instructions to actuate at least one haptic motor 234 of the wearable haptic rig 228 and provide haptic feedback to the player. The haptic feedback may indicate that the explosion occurred in a direction relative to the player avatar. The strength of the haptic feedback may indicate the intensity of the explosion. A distortion effect may be simulated by the at least one haptic motor 234. In an illustrative example, the haptic feedback may be strongest on the chest of the player to indicate the explosion occurred in front of the player avatar. Weaker haptic feedback may be applied to the left and right side of the player to simulate the distortion or echo of the explosion's shockwave as it bounces off the stone hallway's walls.

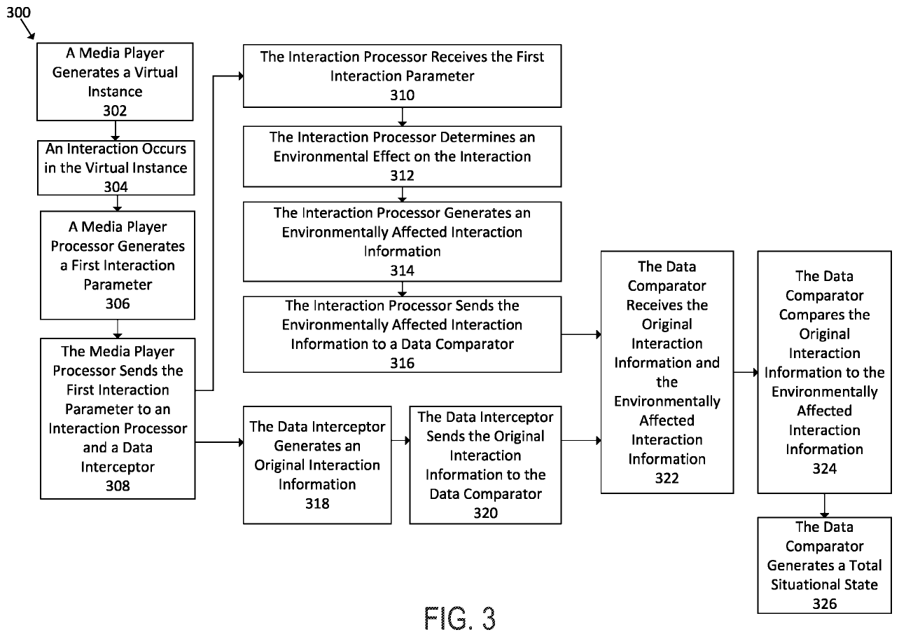

FIG. 3 illustrates a flow chart of an embodiment of the haptic feedback system 300 generating a total situational state 326 according to the disclosure. The media player generates the virtual instance 302 such as a game level. The interaction occurs in the virtual instance 304 and the media player generates a first interaction parameter 306. The media player processor sends the first interaction parameter to the interaction processor and the data interceptor 308.

The interaction processor receives the first interaction parameter 310 and determines an environmental effect on the interaction 312. As discussed hereinbefore, the interaction processor may trace the interaction throughout the virtual environment until it reaches the player to determine what, if any, environmental effects should be applied to the first interaction parameter. The interaction processor applies the environmental effects to the first interaction parameter to generate an environmentally affected interaction information 314. The environmentally affected interaction information is sent to the data comparator 316.

The data interceptor receives the first interaction parameter from the media player processor and generates an original interaction information 318. The original interaction information is sent to the data comparator 320.

The data comparator receives the original interaction information and the environmentally affected interaction information 322. The original interaction information and the environmentally affected interaction information are compared 324 and the environmental effect(s) applied to the first interaction parameter are identified as an environmental parameter. In an alternative embodiment, the interaction processor may store the environmental effect(s) applied to the first interaction parameter as the environmental parameter. The data comparator generates a total situational state 326 using the original interaction information, the environmental parameter, and the environmentally affected interaction information.

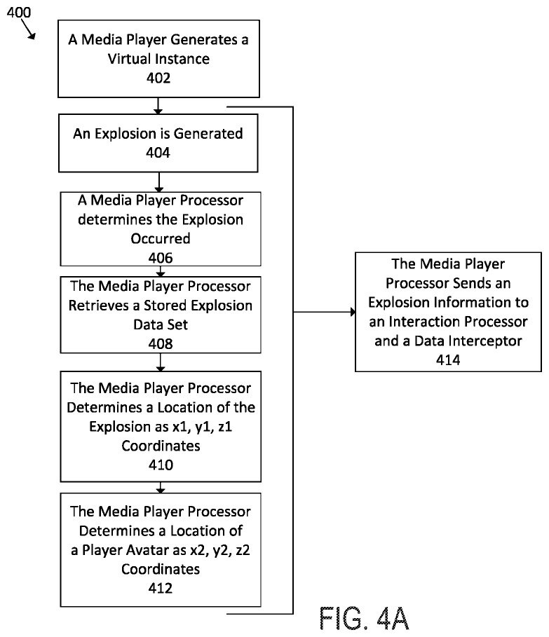

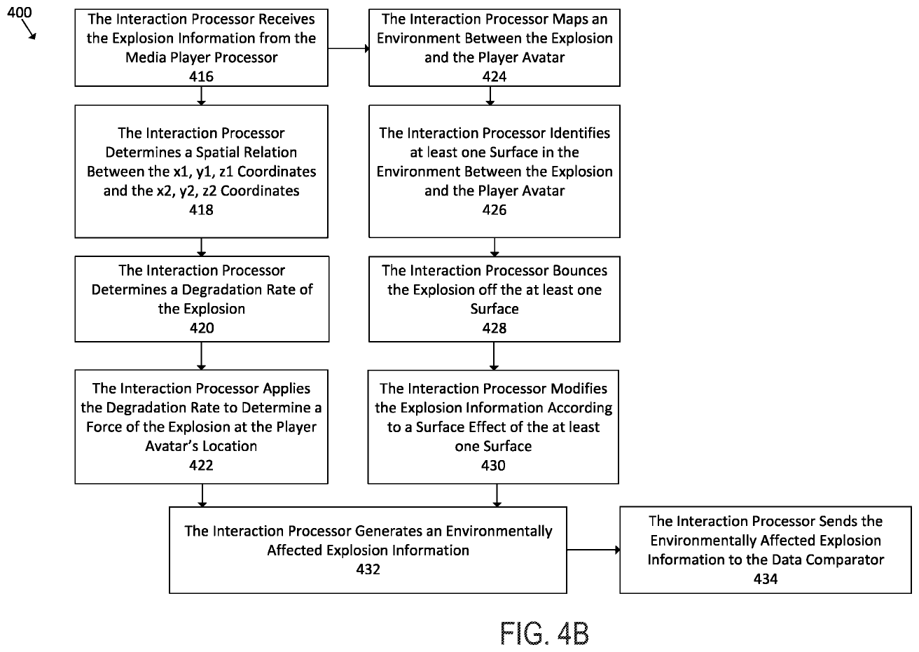

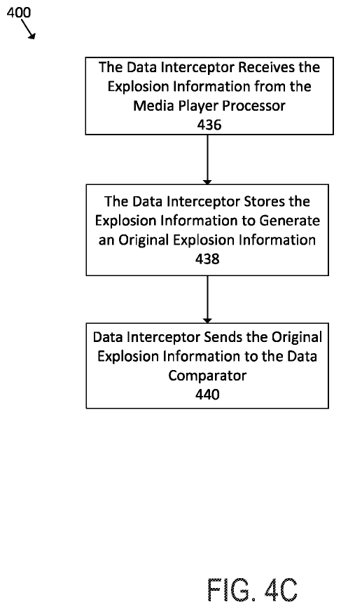

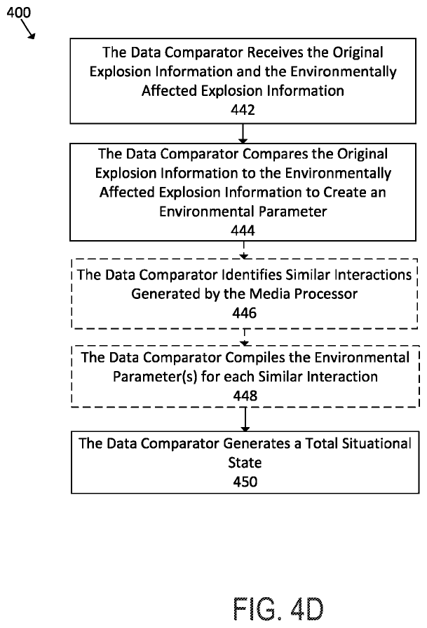

FIG. 4A is an illustrative embodiment of the media player processor of the haptic feedback system 400 generating a first interaction parameter according to the disclosure. The media player may generate a virtual instance 402. An interaction of an explosion is generated within the virtual instance 404. The media player processor determines the explosion occurred 406 and retrieves a stored explosion data set 408. As discussed with respect to FIG. 2, the stored explosion data set may include at least one tag with a unique identifier keyed to the interaction of the explosion. The media player processor may determine a location of the explosion using a spatial coordinate system such as the cartesian coordinate system 410. The media player processor may determine a location of the player avatar within the virtual instance using the same coordinate system 412. The cartesian coordinates of the explosion may be determined as x1, y1, and z1 and the player avatar's coordinates as x2, y2, z2. The cartesian coordinates are grouped with the stored explosion data set and exported as an explosion information to the interaction processor and the data interceptor 414.

FIG. 4B is an illustrative embodiment of the interaction processor of the haptic feedback system 400 generating an environmentally affected interaction information based on the first interaction parameter of FIG. 4A according to the disclosure. The interaction processor receives the explosion information from the media player processor 416. The interaction processor may determine a spatial relation between the explosion and the player avatar by comparing the x1, y1, z1 and the x2, y2, z2 coordinates 418. The interaction processor may determine a degradation rate of the explosion 420, for example in FIG. 2 an example degradation rate of 30% over a distance. That is to say, as the shockwave moves from the epicenter of the explosion it may reduce in strength at a rate of 30% per meter. The interaction processor, having determined a distance between the explosion and the player avatar, can calculate how long the shockwave will take to reach the player. The interaction processor applies the degradation rate to determine the force of the explosion once it reaches the player avatar's location 422.

The interaction processor may map an environment of the virtual instance between the explosion and the player avatar 424. The interaction processor may identify at least one surface or object in the environment between the explosion and the player avatar such as a stone hallway or a door 426. The interaction processor “bounces” the explosion off the at least one surface and traces the explosion shockwave's path to the player avatar 428. The explosion processor may modify the explosion information according to a surface effect of the at least one surface 430. For example, the stone hallway may create a distortion effect such as an echo. Similarly, the strength of the shockwave, determined using the degradation rate, may be reduced due to an obstruction effect caused by the door if the door is located between the player avatar and the explosion. The interaction processor generates an environmentally affected explosion information from the modified explosion information 432 and sends the environmentally affected explosion information to the data comparator 434. In an embodiment, the interaction processor may save the modifications made to the explosion information as an environmental parameter without departing from the disclosure.

FIG. 4C is an illustrative embodiment of the data interceptor of the haptic feedback system 400 generating an original interaction information based on the first interaction parameter of FIG. 4A according to the disclosure. The data interceptor receives the explosion information from the media player processor 436 and stores the explosion information to generate an original explosion information 438. The data interceptor sends the original explosion information to the data comparator 440. In an embodiment, the data interceptor may be implemented as a memory connected to the media player for receiving and storing the explosion information from the media player processor.

FIG. 4D is an illustrative embodiment of the data comparator of the haptic feedback system 400 generating a total situational state based on the environmentally affected interaction information of FIG. 4B and the original interaction information of FIG. 4C according to the disclosure. The data comparator receives the original explosion information from the data interceptor and the environmentally affected explosion information from the interaction processor 442. The data comparator compares the original explosion information to the environmentally affected explosion information to create an environmental parameter 444. In an embodiment, the data comparator may identify similar interactions generated by the media processor 446. For example, the similar interactions may be the same type of interaction or occur within the same location/time period. The data comparator compiles the environmental parameter(s) for each similar interaction 448. The data comparator generates a total situational state 450 including the original explosion information, the environmental parameter, and the environmentally affected explosion information.

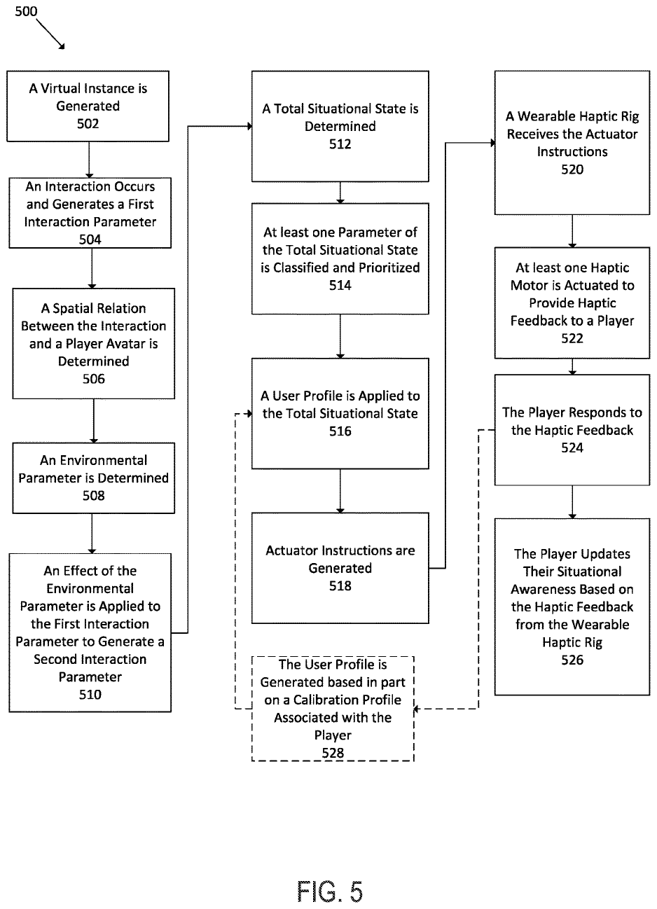

FIG. 5 is a block diagram illustrating a method of providing a haptic based immersive experience 500 according to the disclosure. The virtual instance is generated 502 and an interaction occurs within the virtual instance to generate a first interaction parameter 504. The spatial relation between the interaction and the player avatar is determined 506 to define a 3D space between the interaction and the player. The environmental parameter is determined 508 by analyzing the 3D space between the interaction and the player for objects or effects that would change the interaction before it reaches the player avatar. The effect of the environmental parameter is applied to the first interaction parameter to generate a second interaction parameter 510. A total situational state is determined 512 based at least in part on the first interaction parameter, the environmental parameter, and the second interaction parameter. The total situational state may include the virtual instance and construct a 3D space around the player avatar and between the player avatar and the interaction. At least one parameter of the total situational state is classified and prioritized 514. A user profile specific to the player may be applied to the total situational state 516.

Actuator instructions are generated 518 and sent to a wearable haptic rig 520. At least one haptic motor of the wearable haptic rig is actuated to provide haptic feedback to the player 522. The player, receiving the haptic feedback generated by the interaction, responds to the haptic feedback 524. In an embodiment, the player may respond by turning to face in the direction of the interaction or by completing an action within the virtual instance. The player may update their situational awareness within the virtual instance based on the haptic feedback from the wearable haptic rig 526. As a result, a DHH player may undergo an immersive experience and feel as though they are within the virtual instance.

The user profile may be generated based in part on a calibration profile associated with the player 528. The user profile is applied to the total situational state 516 and may act as a filter to prevent excessive “noise” being provided to the player as haptic feedback. For example, the classification and prioritization of step 514 may remove signals the player wouldn't be aware of. However, if multiple interactions are occurring around the player avatar at once, the player may become overwhelmed or desensitized if they receive all the haptic feedback at the same time. The user profile may be calibrated to include the player's sensitivity to determine how many signals or how much haptic feedback may be provided. Additionally, the user profile may include user settings for how the user prefers to receive the haptic feedback such as the duration of the haptic feedback. The user may set a haptic feedback preference for the haptic feedback to be provided in long pulses, short bursts, etc. The calibration profile may be set using a calibration scenario or may monitor the player during play to update the user profile continuously or at regular intervals. For example, a response time of the player may be monitored to update the calibration profile. If the response time changes, it may indicate the player can handle more or fewer signals/haptic feedback at the same time. The calibration profile may be updated accordingly and cause the generated user profile 528 to filter out more or fewer signals from the total situational state 516.

In another aspect of the present disclosure, a non-transitory computer-readable medium with non-transitory program code recorded thereon is disclosed. The program code is executed by a processor and includes program code to generate a virtual instance 502. The program code still further includes program code to generate an interaction having a first interaction parameter within the virtual instance 504. The program code also includes program code to determine a spatial relation between a player avatar and the interaction 506. The program code further includes program code to analyze an environment of the virtual instance and determine at least one environmental parameter affecting the first interaction parameter 508. The program code still further includes program code to apply the environmental parameter to the first interaction parameter and generate a second interaction parameter 510. The program code also includes program code to generate a total situational state including the virtual instance, the first interaction parameter, and the second interaction parameter 512. The program code further includes program code to classify and prioritize at least one parameter of the total situational state 514. The program code still further includes program code to apply the user profile to the total situational state 516. The program code also includes program code to generate the actuator instructions and send the actuator instructions to the wearable haptic rig 518. The program code further includes program code to apply the actuator instructions to at least one haptic motor of the wearable haptic rig 520. The program code still further includes program code to actuate the at least one haptic motor to provide a haptic feedback 522.

The memory of the processor, which may be internal memory and/or external memory, may be implemented in firmware and/or software implementation. The firmware and/or software implementation methodologies may be implemented with modules (e.g., procedures, functions, and so on) that perform the functions described herein. A machine-readable medium tangibly embodying instructions may be used in implementing the methodologies described herein. For example, software codes may be stored in a memory and executed by a processor unit (e.g., media player processor 204). Memory may be implemented within the processor unit or external to the processor unit. As used herein, the term “memory” refers to types of long term, short term, volatile, nonvolatile, or other memory and is not to be limited to a particular type of memory or number of memories, or type of media upon which memory is stored.

If implemented in firmware and/or software, the functions may be stored as one or more instructions or code on a computer-readable medium. Examples include computer-readable media encoded with a data structure and computer-readable media encoded with a computer program. Computer-readable media includes physical computer storage media. A storage medium may be an available medium that can be accessed by a computer. By way of example, and not limitation, such computer-readable media can include RAM, ROM, EEPROM, CD-ROM or other optical disk storage, magnetic disk storage or other magnetic storage devices, or other medium that can be used to store desired program code in the form of instructions or data structures and that can be accessed by a computer; disk and disc, as used herein, includes compact disc (CD), laser disc, optical disc, digital versatile disc (DVD), floppy disk and Blu-ray disc where disks usually reproduce data magnetically, while discs reproduce data optically with lasers. Combinations of the above should also be included within the scope of computer-readable media.

In addition to storage on computer readable medium, instructions and/or data may be provided as signals on transmission media included in a communication apparatus. For example, a communication apparatus may include a transceiver having signals indicative of instructions and data. The instructions and data are configured to cause one or more processors to implement the functions outlined in the claims.

Although the present disclosure and its advantages have been described in detail, it should be understood that various changes, substitutions and alterations can be made herein without departing from the technology of the disclosure as defined by the appended claims. For example, relational terms, such as “above” and “below” are used with respect to components. Of course, if the component is inverted, above becomes below, and vice versa. Additionally, if oriented sideways, above and below may refer to sides of a component. Moreover, the scope of the present application is not intended to be limited to the particular configurations of the process, machine, manufacture, composition of matter, means, methods and steps described in the specification. As one of ordinary skill in the art will readily appreciate from the disclosure, processes, machines, manufacture, compositions of matter, means, methods, or steps, presently existing or later to be developed that perform substantially the same function or achieve substantially the same result as the corresponding configurations described herein may be utilized according to the present disclosure. Accordingly, the appended claims are intended to include within their scope such processes, machines, manufacture, compositions of matter, means, methods, or steps.

Those of skill would further appreciate that the various illustrative logical blocks, modules, circuits, and algorithm steps described in connection with the disclosure herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure.

The various illustrative logical blocks, modules, and circuits described in connection with the disclosure herein may be implemented or performed with a general-purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices, e.g., a combination of a DSP and a microprocessor, multiple microprocessors, one or more microprocessors in conjunction with a DSP core, or any other such configuration.

The steps of a method or algorithm described in connection with the disclosure may be embodied directly in hardware, in a software module executed by a processor, or in a combination of the two. A software module may reside in RAM, flash memory, ROM, EPROM, EEPROM, registers, hard disk, a removable disk, a CD-ROM, or any other form of storage medium known in the art. An exemplary storage medium is coupled to the processor such that the processor can read information from, and write information to, the storage medium. In the alternative, the storage medium may be integral to the processor. The processor and the storage medium may reside in an ASIC. The ASIC may reside in a user terminal. In the alternative, the processor and the storage medium may reside as discrete components in a user terminal.

In one or more exemplary designs, the functions described may be implemented in hardware, software, firmware, or any combination thereof. If implemented in software, the functions may be stored on or transmitted over as one or more instructions or code on a computer-readable medium. Computer-readable media includes both computer storage media and communication media including any medium that facilitates transfer of a computer program from one place to another. A storage media may be any available media that can be accessed by a general purpose or special purpose computer. By way of example, and not limitation, such computer-readable media can include RAM, ROM, EEPROM, CD-ROM or other optical disk storage, magnetic disk storage or other magnetic storage devices, or any other medium that can be used to carry or store specified program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, includes compact disc (CD), laser disc, optical disc, digital versatile disc (DVD), floppy disk and Blu-ray disc where disks usually reproduce data magnetically, while discs reproduce data optically with lasers. Combinations of the above should also be included within the scope of computer-readable media.

It should be understood that when an element is referred to as being “connected” or “coupled” to another element (or variations thereof), it can be directly connected or coupled to the other element or intervening elements may be present. In contrast, when an element is referred to as being “directly connected” or “directly coupled” to another element (or variations thereof), there are no intervening elements present.

Benefits, other advantages, and solutions to problems have been described herein with regard to specific embodiments. However, the benefits, advantages, solutions to problems, and elements that may cause any benefit, advantage, or solution to occur or become more pronounced are not to be construed as critical, required, or essential features or elements of the disclosure. It should be appreciated that in the appended claims, reference to an element in the singular is not intended to mean “one and only one” unless explicitly so stated, but rather “one or more.”

Embodiments of the present disclosure are described herein with reference to the accompanying drawings. However, the present disclosure should not be construed as limited to the embodiments set forth herein. Rather, these embodiments are provided so that this disclosure will be thorough and complete, and will fully convey the scope of the present disclosure to those skilled in the art. As used herein, the singular forms “a,” “an,” and “the” are intended to include the plural forms as well, unless the context clearly indicates otherwise. It will be further understood that the terms “comprises,” “comprising,” “having,” “includes,” “including,” and/or variations thereof, when used herein, specify the presence of stated features, steps, operations, elements, and/or components, but do not preclude the presence or addition of one or more other features, steps, operations, elements, components, and/or groups thereof.

The description of the disclosure is provided to enable any person skilled in the art to make or use the disclosure. Various modifications to the disclosure will be readily apparent to those skilled in the art, and the generic principles defined herein may be applied to other variations without departing from the spirit or scope of the disclosure. Thus, the disclosure is not intended to be limited to the examples and designs described herein but is to be accorded the widest scope consistent with the principles and novel features disclosed herein.

Although illustrative embodiments of the present disclosure have been described herein with reference to the accompanying drawings, it is to be understood that the present disclosure is not limited to those precise embodiments, and that various other changes and modifications may be made by one skilled in the art without departing from the scope or spirit of the disclosure.

What is claimed is:

1. A method of providing haptic feedback in a virtual environment comprising:
generating a virtual instance;
generating an interaction within the virtual instance, the interaction including a first interaction parameter;
determining a spatial relation between a player avatar and the interaction;
analyzing an environment of the virtual instance and determining an environmental parameter affecting the interaction;
applying the environmental parameter to the first interaction parameter and generating a second interaction parameter;
generating a total situational state including the virtual instance, the first interaction parameter, and the second interaction parameter;
classifying and prioritizing at least one parameter of the total situational state;
applying a user profile to the total situational state;
generating at least one actuator instruction based at least in part on the total situational state and sending the at least one actuator instruction to a wearable haptic rig;
applying the at least one actuator instruction to at least one haptic motor of the wearable haptic rig; and
actuating the at least one haptic motor to provide a haptic feedback to a player.

2. The method for providing haptic feedback in a virtual environment of claim 1, further comprising calibrating the user profile based at least in part on a player's situational awareness parameters.

3. The method for providing haptic feedback in a virtual environment of claim 1, further comprising generating a classification and prioritization table.

4. The method for providing haptic feedback in a virtual environment of claim 1, further comprising ranking the interaction against at least one other interaction within the virtual instance.

5. The method for providing haptic feedback in a virtual environment of claim 1, further comprising generating a calibration profile using a calibration scenario.

6. The method for providing haptic feedback in a virtual environment of claim 5, further comprising filtering the interaction using a user situational filter generated using the calibration profile.

7. The method for providing haptic feedback in a virtual environment of claim 5, further comprising updating the user situational filter based in part on variables of the player monitored during play.

8. An apparatus for providing haptic feedback in a virtual environment, comprising:
a memory; and
at least one processor coupled to the memory, the at least one processor configured:
to generate a virtual instance;
to generate an interaction within the virtual instance, the interaction including a first interaction parameter;
to determine a spatial relation between a player avatar and the interaction;
to analyze an environment of the virtual instance and determine at least one environmental parameter affecting the interaction;
to apply the environmental parameter to the first interaction parameter and generate a second interaction parameter;
to generate a total situational state including the virtual instance, the first interaction parameter, and the second interaction parameter;
to classify and prioritize at least one parameter of the total situational state;
to apply a user profile to the total situational state;
to generate at least one actuator instruction and send the at least one actuator instruction to a wearable haptic rig;
to apply the actuator instruction to at least one haptic motor of the wearable haptic rig; and
to actuate the at least one haptic motor to provide a haptic feedback.

9. The apparatus for providing haptic feedback in a virtual environment of claim 8, in which the at least one processor is further configured to calibrate the user profile based at least in part on a player's situational awareness parameters.

10. The apparatus for providing haptic feedback in a virtual environment of claim 8, in which the at least one processor is further configured to generate a classification and prioritization table.

11. The apparatus for providing haptic feedback in a virtual environment of claim 8, in which the at least one processor is further configured to rank at least one other interaction within the virtual instance.

12. The apparatus for providing haptic feedback in a virtual environment of claim 8, in which the at least one processor is further configured to generate a calibration profile using a calibration scenario.

13. The apparatus for providing haptic feedback in a virtual environment of claim 12, in which the at least one processor is further configured to filter the interaction using a user situational filter generated using the calibration profile.

14. The apparatus for providing haptic feedback in a virtual environment of claim 12, in which the at least one processor is further configured to update the user situational filter based in part on variables of the player monitored during play.

15. The apparatus for providing haptic feedback in a virtual environment of claim 8, in which the at least one processor is further configured to apply the actuator instruction to the at least one haptic motor of the wearable haptic rig according to a haptic feedback preference.

16. A non-transitory computer-readable medium having program code recorded thereon, the program code executed by a processor and comprising:
program code to generate a virtual instance;
program code to generate an interaction within the virtual instance, the interaction including a first interaction parameter;
program code to determine a spatial relation between a player avatar and the interaction;
program code to analyze an environment of the virtual instance and determine at least one environmental parameter affecting the interaction;
program code to apply the environmental parameter to the first interaction parameter and generate a second interaction parameter;
program code to generate a total situational state including the virtual instance, the first interaction parameter, and the second interaction parameter;
program code to classify and prioritize at least one parameter of the total situational state;
program code to apply a user profile to the total situational state;
program code to generate at least one actuator instruction and send the at least one actuator instruction to a wearable haptic rig;
program code to apply the at least one actuator instruction to at least one haptic motor of the wearable haptic rig; and
program code to actuate the at least one haptic motor to provide a haptic feedback.

17. The non-transitory computer-readable medium of claim 16, in which the program code comprises program code to calibrate the user profile based at least in part on a player's situational awareness parameters.

18. The non-transitory computer-readable medium of claim 16, in which the program code comprises program code to generate a classification and prioritization table and determine whether to provide haptic feedback to the player based in part on a ranking of the interaction on the classification and prioritization table.

19. The non-transitory computer-readable medium of claim 16, in which the program code comprises program code to generate a calibration profile using a calibration scenario.

20. The non-transitory computer-readable medium of claim 16, in which the program code comprises program code to update a user situational filter generated using the calibration scenario based in part on variables of the player monitored during play.
